(19) United States
(12) Patent Application Publication
(10) Pub. No.: US 2025/0143687 A1
(43) Pub. Date: May 8, 2025

(54) TISSUE REPAIR SYSTEMS AND SURGICAL METHODS

(71) Applicant: ARTHREX, INC., Naples, FL (US)

(72) Inventors: Thomas Dooney, JR., Naples, FL (US); Peter J. Dreyfuss, Naples, FL (US)

(21) Appl. No.: 18/935,733

(22) Filed: Nov. 4, 2024

Related U.S. Application Data
(60) Provisional application No. 63/597,086, filed on Nov. 8, 2023.

(51) Int. Cl. A61B 17/04

(52) U.S. Cl. CPC A61B 17/0401; A61B 2017/0404

(57) ABSTRACT

Tissue repair systems and methods are provided for reducing and fixating tissue to bone. The proposed systems and methods utilize one or more suture anchors and one or more surgical buttons for reducing and fixating the tissue to the bone. Additional suture anchors may be used as part of a multi-row fixation technique for providing a desired area of footprint compression over top of the tissue.

### CROSS REFERENCE TO RELATED APPLICATIONS

This application claims the benefit of U.S. Provisional Application No. 63/597,086, which was filed on Nov. 8, 2023 and is incorporated herein by reference in its entirety.

### BACKGROUND

This disclosure relates to the field of surgery, and more particularly to systems and associated surgical methods for reducing, fixating, and compressing tissue relative to bone.

Repetitive trauma to a joint, such as a knee, ankle, hip, or shoulder joint, for example, may result in the development of tissue defects (e.g., soft tissue tears, cartilage defects, etc.). If not treated, tissue defects could further deteriorate, thereby causing joint instability and discomfort.

### SUMMARY

This disclosure relates to tissue repair systems and methods for reducing and fixating tissue to bone. The proposed systems and methods utilize one or more suture anchors and one or more buttons for reducing and fixating the tissue to the bone. Additional suture anchors may be used as part of a multi-row fixation technique for providing a desired area of footprint compression over top of the tissue.

An exemplary surgical method may include, inter alia, inserting a first suture anchor into a bone, passing a first suture of the first suture anchor through a tissue, feeding the first suture through a surgical button, securing the first suture relative to the surgical button, inserting a second suture anchor into the bone at a position that is lateral to the first suture anchor, feeding a second suture of the second suture anchor through the surgical button at a different location than the first suture, and securing the second suture relative to the surgical button.

Another exemplary surgical method may include, inter alia, inserting a first suture anchor into a bone, passing a first suture of the first suture anchor through a tissue, feeding the first suture through a surgical button, tensioning the first suture to firmly fixate the surgical button against the tissue, inserting a second suture anchor into the bone at a position that is lateral to the first suture anchor, looping a second suture of the second suture anchor about third suture of the surgical button, and securing the second suture back to the second suture anchor.

An exemplary tissue repair system may include, inter alia, a suture anchor and a surgical button. The surgical button includes a first opening configured as a first round hole, a second opening configured as a second round hole, and a third opening configured as a semi-circular or arch-shaped slot.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 schematically illustrates a step of surgical method for reattaching torn tissue to bone.

FIG. 2 illustrates another step of a surgical method for reattaching torn tissue to bone.

FIGS. 3 and 4 illustrate an exemplary knotless suture anchor.

FIGS. 5 and 6 illustrate another exemplary knotless suture anchor. FIG. 7 illustrates another step of a surgical method for reattaching torn tissue to

bone.

FIG. 8 illustrates another step of a surgical method for reattaching torn tissue to bone.

FIG. 9 illustrates another step of a surgical method for reattaching torn tissue to bone.

FIG. 10 illustrates another step of a surgical method for reattaching torn tissue to bone.

FIG. 11 illustrates another step of a surgical method for reattaching torn tissue to bone.

FIG. 12 illustrates yet another step of a surgical method for reattaching torn tissue to bone.

FIG. 13 illustrates a final repair construct of a surgical method for reattaching tissue to bone.

FIGS. 14 and 15 illustrate yet another exemplary knotless suture anchor.

FIGS. 16 and 17 illustrate exemplary surgical buttons for use as part of a surgical method for reattaching tissue to bone.

FIG. 18 schematically illustrates an exemplary tissue fixation system.

### DETAILED DESCRIPTION

This disclosure is directed to tissue repair systems and surgical methods for repairing tissue defects within a joint. The system and methods described herein may be utilized to reduce, fixate, and compress tissue to bone. These and other features of this disclosure are described in further detail below.

An exemplary surgical method may include, inter alia, inserting a first suture anchor into a bone, passing a first suture of the first suture anchor through a tissue, feeding the first suture through a surgical button, securing the first suture relative to the surgical button, inserting a second suture anchor into the bone at a position that is lateral to the first suture anchor, feeding a second suture of the second suture anchor through the surgical button at a different location than the first suture, and securing the second suture relative to the surgical button.

In a further embodiment, a first opening of the surgical button is a first round hole, a second opening of the surgical button is a second round hole, and a third opening of the surgical button is a semi-circular or arch-shaped slot.

In a further embodiment, the first round hole, the second round hole, and the semi-circular or arch-shaped slot are arranged in a smiley face pattern.

In a further embodiment, securing the first suture relative to the surgical button includes splicing the first suture through itself to establish a mattress stitch that spans between a first opening and a second opening of the surgical button.

In a further embodiment, securing the first suture relative to the surgical button further includes tensioning the first suture to pull the tissue laterally over top of the first suture anchor.

In a further embodiment, securing the first suture relative to the surgical button further includes tensioning the first suture to firmly fixate the surgical button against the tissue.

In a further embodiment, securing the second suture relative to the surgical button includes splicing the second suture through itself to establish a suture loop that is looped through the surgical button.

In a further embodiment, securing the second suture relative to the surgical button further includes tensioning the second suture to reduce the second suture further into place and provide a desired area of footprint compression over top of the tissue.

In a further embodiment, the first suture anchor is part of a medial row of suture anchors and the second suture anchor is part of a lateral row of suture anchors, and a crisscross suture pattern establishes a desired area of footprint compression over top of the tissue.

In a further embodiment, the first suture anchor and the second suture anchor are made exclusively of soft, suture-based materials.

Another exemplary surgical method may include, inter alia, inserting a first suture anchor into a bone, passing a first suture of the first suture anchor through a tissue, feeding the first suture through a surgical button, tensioning the first suture to firmly fixate the surgical button against the tissue, inserting a second suture anchor into the bone at a position that is lateral to the first suture anchor, looping a second suture of the second suture anchor about a third suture of the surgical button, and securing the second suture back to the second suture anchor.

In a further embodiment, the third suture is a suture loop construct that is attached to the surgical button.

In a further embodiment, the third suture is a suture loop construct that is integrally formed with the surgical button.

In a further embodiment, tensioning the first suture includes splicing the first suture through itself to establish a mattress stitch that spans between a first opening and a second opening of the surgical button.

In a further embodiment, tensioning the first suture pulls the tissue laterally over top of the first suture anchor.

In a further embodiment, securing the second suture back to the second suture anchor includes splicing the second suture through itself to establish a suture loop that is looped around the third suture of the surgical button.

In a further embodiment, securing the second suture back to the second suture anchor includes tensioning the second suture to reduce the second suture further into place and provide a desired area of footprint compression over top of the tissue.

In a further embodiment, the surgical method may include inserting a third suture anchor into the bone, passing a fourth suture of the third suture anchor through the tissue, feeding the fourth suture through a second surgical button, and tensioning the fourth suture to firmly fixate the second surgical button against the tissue.

In a further embodiment, the surgical method may include inserting a fourth suture anchor into the bone at a position that is lateral to the third suture anchor, looping a fifth suture of the fourth suture anchor about a sixth suture of the second surgical button, and securing the fifth suture back to the fourth suture anchor.

An exemplary tissue repair system may include, inter alia, a suture anchor and a surgical button. The surgical button includes a first opening configured as a first round hole, a second opening configured as a second round hole, and a third opening configured as a semi-circular or arch-shaped slot.

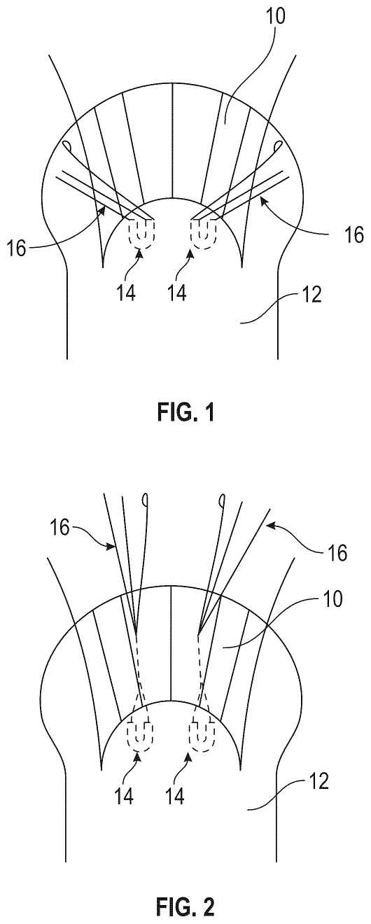

FIGS. 1-17 schematically illustrate various aspects associated with a surgical method for attaching a tissue 10 (e.g., ligament, tendon, graft, etc.) to a bone 12. The tissue 10 may have torn away from the bone 12 during vigorous exercise or sporting activities, for example. When such tears occur, reattachment is often necessary to repair the tissue defect. Although the surgical method is described herein for reapproximating and fixating torn tissue back to bone, the surgical methods of this disclosure could be utilized to repair any type of tissue effect.

The surgical method schematically illustrated in FIGS. 1-17 could be used in conjunction with a variety of orthopedic surgical repairs, including but not limited to rotator cuff repairs, Achilles tendon repairs, patellar tendon repairs, ACL/PCL reconstructions, hip and shoulder reconstructions, among many others. The bone 12 may therefore be associated with any joint of the human musculoskeletal system (e.g., shoulder, knee, hip, ankle, etc.).

In an embodiment, the surgical method is performed as an arthroscopic procedure by working through various arthroscopic portals. However, the exemplary surgical method could alternatively be performed as an open procedure within the scope of this disclosure. As detailed below, the exemplary surgical method may be employed to reduce and then reattach the tissue 10 to the bone 12 in a manner that enhances footprint compression to maximize tissue-to-bone contact.

Referring first to FIG. 1, after appropriately preparing the bone 12 (e.g., by debriding, creating a bleeding bone bed, preparing bone sockets, etc.), a medial row of fixation devices may be implanted into the bone 12. The medial row of fixation devices may include one or more suture anchors 14. In an embodiment, the suture anchors 14 are knotless suture anchors that do not require the need to tie any knots in the various structures for reducing and securing the tissue 10 to the bone 12. However, traditional knot tying style suture anchors could alternatively or additionally be used in the medial row of fixation devices.

In an embodiment, the suture anchors 14 of the medial row are placed at the articular margin of the bone 12. However, other implantation locations could be selected based on the performing surgeon's own preferences. Notably, although two suture anchors 14 are illustrated as being part of the medial row in the illustrated embodiment, a greater or fewer number of suture anchors could be utilized as part of the surgical method within the scope of this disclosure. For example, the medial row could include but a single suture anchor 14.

Each suture anchor 14 may be pre-loaded with one or more sutures 16. The sutures 16 may include individual suture strands, multiple suture strands, suture tape, or any other suture-like product. As shown in FIG. 2, the sutures 16 may be passed upwardly through the tissue 10 after each suture anchor 14 is adequately fixated within the bone 12. At this stage of the surgical method, the tissue 10 is not directly over top of the suture anchors 14.

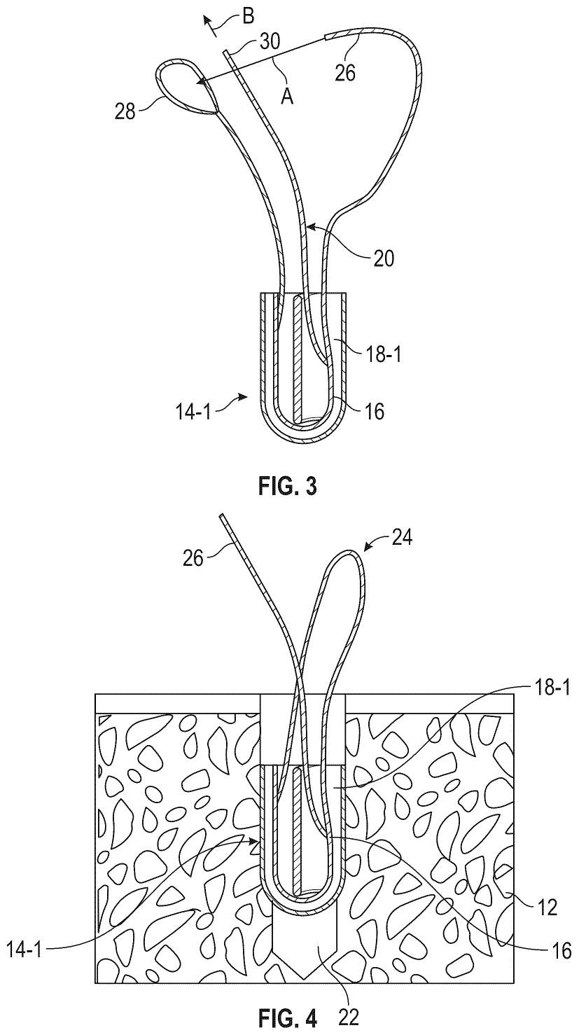

FIGS. 3-6 illustrate exemplary knotless suture anchors that can be utilized as the suture anchors 14 of the medial row of fixation devices when performing the surgical methods described herein. Knotless suture anchors similar to those shown in FIGS. 3-6 may be utilized either alone or in combination with one another as part of the medial row during the surgical method.

An exemplary knotless suture anchor 14-1 is illustrated in FIGS. 3 and 4. In this embodiment, the knotless suture anchor 14-1 is a “soft” anchor assembly made exclusively of soft, suture-based materials. The suture-based materials may include soft materials such as yarns, fibers, filaments, strings, fibrils, strands, sutures, etc., or any combination of such materials. The soft materials may be synthetic or natural materials, or combinations of synthetic and natural materials, and may be bio-degradable or non-degradable within the scope of this disclosure. The soft, suture-based materials confer the knotless suture anchor 14-1 with the ability to be inserted into bone sockets/holes and bunch together, collapse, expand and/or change shape to fixate within the socket/hole.

The knotless suture anchor 14-1 may include an anchor body 18-1 and a suture 16 received through the anchor body 18-1. A shuttle device 20 may be spliced through portions of the suture 16. The shuttle device 20 may be a passing wire or another suture, for example.

The anchor body 18-1 of the knotless suture anchor 14-1 may be inserted into a socket 22 formed in the bone 12 (see FIG. 4). The socket 22 may be a preformed opening formed in the bone 12 that is configured for receiving the anchor body 18-1.

The shuttle device 20 may be pre-assembled to the suture 16 as shown in FIG. 3, and the suture 16 may form a suture loop 24 after the suture 16 is shuttled through itself as shown in FIG. 4. For example, a suture tail 26 of the suture 16 may be passed through an eyelet 28 of the shuttle device 20 (in the direction of arrow A of FIG. 3), and then a free end 30 of the shuttle device 20 may be pulled (in the direction of arrow B of FIG. 3) to allow the suture 16 to pass through itself and form the suture loop 24. The perimeter of the suture loop 24 is adjustable to allow the construct to be self-cinching and to adjust the tension on the construct that is to be fixated.

In an exemplary embodiment, the suture anchors 14 of the medial row may include the design of the knotless suture anchor 14-1 for performing the surgical method steps herein. However, other type of suture anchors or combinations of suture anchors are contemplated within the scope of this disclosure.

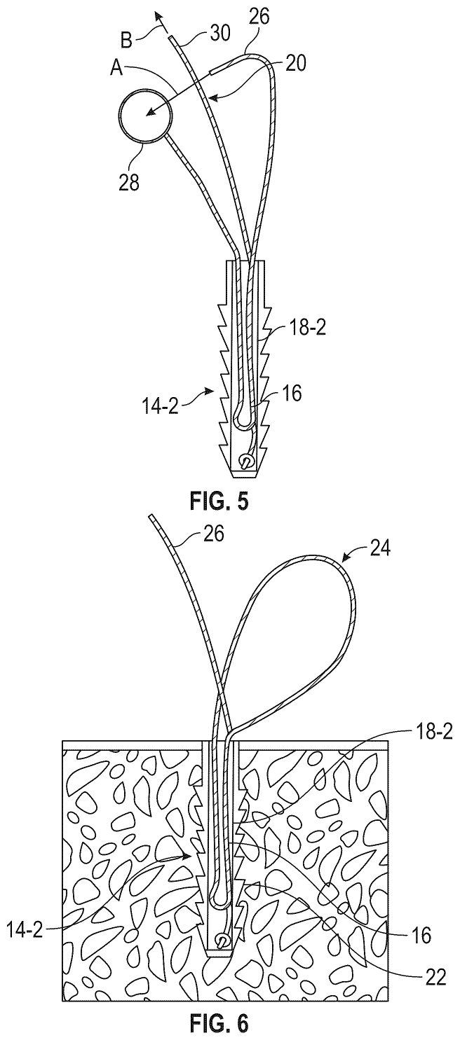

Another exemplary knotless suture anchor 14-2 is illustrated in FIGS. 5 and 6. The knotless suture anchor 14-2 may include an anchor body 18-2 and a suture 16 received through the anchor body 18-2. In this embodiment, the anchor body 18-2 is a relatively rigid plastic body and thus the knotless suture anchor 14-2 is not considered to be a “soft” anchor assembly.

The knotless suture anchor 14-2 may include a tensionable knotless mechanism that is similar to that of the knotless suture anchor 14-1. For example, a shuttle device 20 may be spliced through portions of the suture 16. The shuttle device 20 may be a passing wire or another suture, for example. The anchor body 18-2 of the knotless suture anchor 14-2 may be inserted into a socket 22 formed in the bone 12 (see FIG. 6). The socket 22 may be a preformed opening formed in the bone 12 that is configured for receiving the anchor body 18-2. The shuttle device 20 may be pre-assembled to the suture 16 as shown in FIG. 5 and may be utilized to form a suture loop 24 after the suture 16 is shuttled through itself as shown in FIG. 6. For example, a suture tail 26 of the suture 16 may be passed through an eyelet 28 of the shuttle device 20 (in the direction of arrow A of FIG. 5), and then a free end 30 of the shuttle device 20 may be pulled (in the direction of arrow B of FIG. 5) to allow the suture 16 to pass through itself and form the suture loop 24. The perimeter of suture loop 24 is adjustable to allow the construct to be self-cinching and to adjust the tension on the construct to be fixated.

In an exemplary embodiment, the suture anchors 14 of the medial row may include the design of the knotless suture anchor 14-2 (rather than that of the knotless suture anchor 14-1, for example) for performing the surgical method steps described herein. However, other type of suture anchors or combinations of suture anchors are contemplated within the scope of this disclosure.

Referring now to FIG. 7, the surgical method depicted in FIGS. 1 and 2 may continue by feeding the suture 16 of each suture anchor 14 through a first opening 32 of a surgical button 34 and by feeding the shuttle device 20 (both free end 30 and eyelet 28, for example) through a second opening 36 of the surgical button 34. In general, the number of surgical buttons 34 utilized during the surgical method will match the number of suture anchors 14 utilized as part of the medial row. The surgical buttons 34 may be embroidered from polyester or some other permanent or absorbable materials, molded/machined from a tissue compatible polymer, etc.

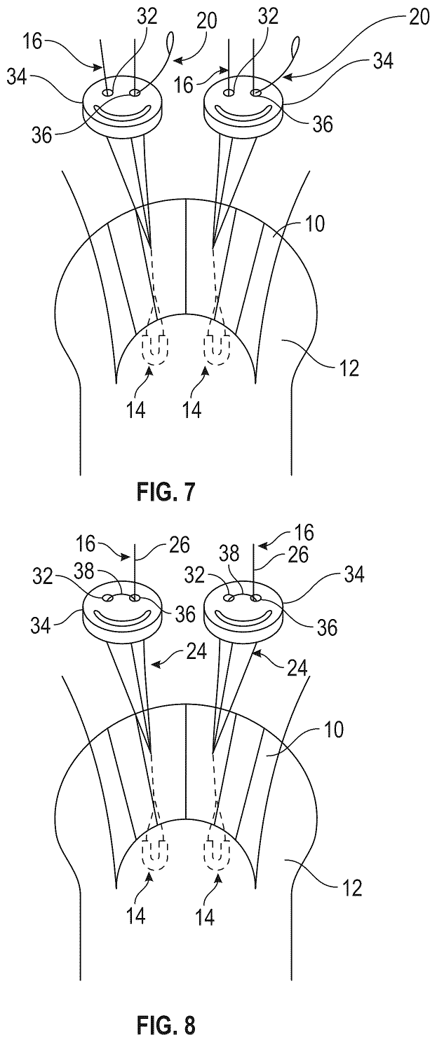

Referring to FIG. 8, each suture 16 may then be spliced through itself using the shuttle device 20 to form the suture loop 24 about the surgical button 34. Splicing the suture 16 through itself in this manner creates a mattress stitch 38 that spans between the first opening 32 and the second opening 36 of the surgical button 34. The mattress stitch 38 may be established by an apex of the suture loop 24, for example.

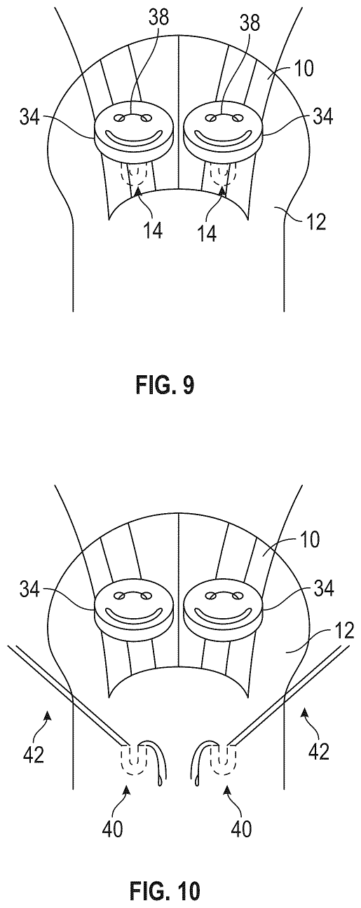

The sutures 16 may then be further tensioned to approximate each surgical button 34 firmly against the tissue 10 (see FIG. 9). The tissue 10 therefore reduces into place over top of the suture anchors 14 (e.g., is pulled laterally) and is fixated against the bone 12. The suture tails 26 of the sutures 16 may be removed (e.g., cut) once tensioning is complete. At this stage of the surgical method, the suture anchors 14 are located underneath the tissue 10 and the surgical buttons 34.

The surgical repair can be considered complete at this point of the surgical method. Alternatively, if double row fixation is desired, the surgical method can proceed by implanting a lateral row of fixation devices into the bone 12. As shown in FIG. 10, the lateral row of fixation devices may include one or more suture anchors 40. In an embodiment, the suture anchors 40 are knotless suture anchors that do not require the need to tie any knots in the various structures for achieving fixation. However, traditional knot tying style suture anchors could alternatively or additionally be used in the lateral row.

In an embodiment, the suture anchors 40 of the lateral row are placed laterally from the medial row of suture anchors 14 and slightly distal to the greater tuberosity of the bone 12. However, other implantation locations could be selected based on the performing surgeon's own preferences and depending on the type of orthopedic procedure being performed. Notably, although two suture anchors 40 are illustrated as being part of the lateral row in the illustrated embodiment, a greater or fewer number of suture anchors could be utilized as part of the surgical method within the scope of this disclosure.

Each suture anchor 40 may be pre-loaded with one or more sutures 42. The sutures 42 may include individual suture strands, multiple suture strands, suture tape, or any other suture-like product.

The suture anchors 40 of the lateral row may include the same or a different design as the suture anchors 14 of the medial row. In an embodiment, the suture anchors 40 of the lateral row may include the design of the knotless suture anchor 14-1. In another embodiment, the suture anchors 40 of the lateral row may include the design of the knotless suture anchor 14-2.

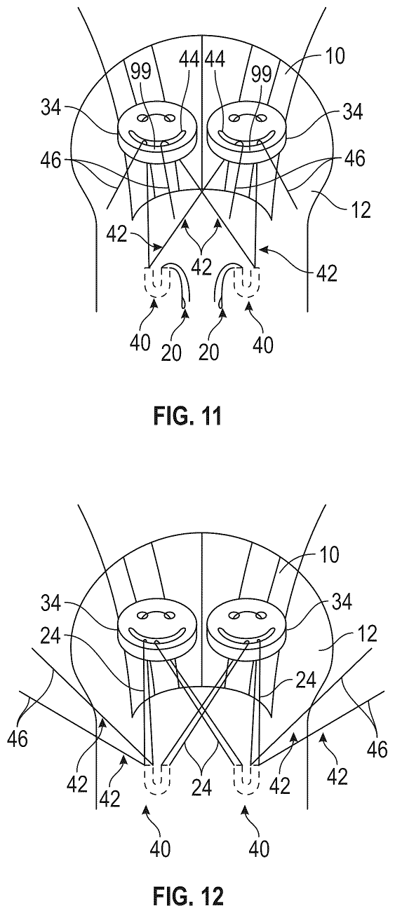

Referring now to FIG. 11, one suture 42 from each suture anchor 40 may be fed through a third opening 44 of each surgical button 34. The sutures 42 may be fed through the third openings 44 either before or after fully approximating the surgical buttons 34 firmly down against the tissue 10. The sutures 42 may be positioned such that a suture tail 46 of each suture 42 is looped over a section 99 of the surgical button 34 that borders the third opening 44.

The third opening 44 may be sized and shaped differently from the first opening 32 and the second opening 36. In an embodiment, the first opening 32 and the second opening 36 are round holes formed through the surgical button 34, and the third opening 44 is a semi-circular or arch-shaped slot formed through the surgical button. Together, the first opening 32, the second opening 36, and the third opening 44 may establish a smiley face pattern within the body of the surgical button 34. However, other configurations are contemplated within the scope of this disclosure.

Referring next to FIGS. 11 and 12, each suture 42 may be secured back to its suture anchor 40 by splicing the suture 42 through itself using the shuttle device 20. The splicing procedure forms the suture loop 24 about the surgical button 34, thereby securing each suture 42 to the surgical button 34. The suture tails 46 may be further tensioned to reduce the sutures 42 further into place and more firmly fixate the tissue 10 against the bone 12. The suture tails 46 of the sutures 16 may be removed (e.g., cut) once tensioning is complete.

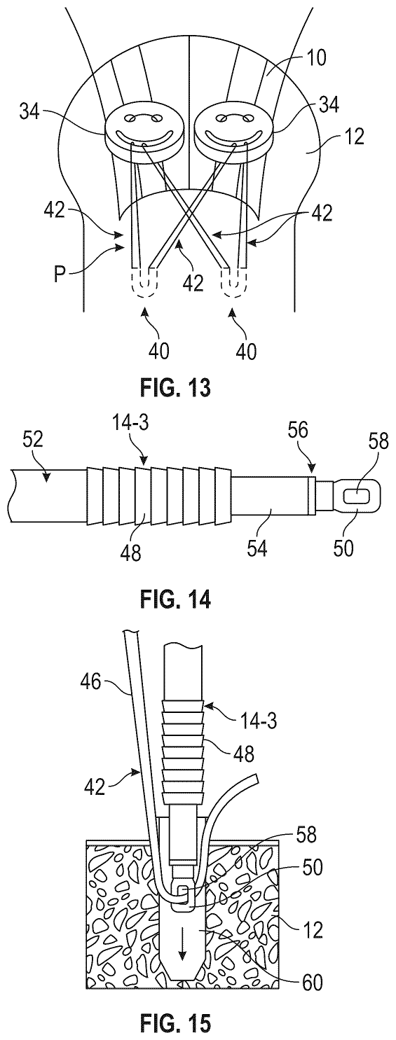

The final construct achieved by the surgical method is shown in FIG. 13. The sutures 42 may be arranged in various bridging configurations between the surgical buttons 34 and the suture anchors 40 of the lateral row. For example, as shown in FIG. 13, the sutures 42 may be arranged in a crisscross pattern P that provides a desired area of footprint compression over top of the tissue 10. Any bridging configuration using any number of suture anchors, surgical buttons, and sutures can be achieved as part of the surgical method.

In the above embodiments, the sutures 42 are connected to the surgical buttons 34 after implanting the lateral row suture anchors 40 into the bone 12. However, other implementations are possible. For example, the suture anchors 40 could be implanted after connecting the sutures 42 to the surgical buttons 34 by employing the design of another exemplary knotless suture anchor 14-3 shown in FIGS. 14-15.

The knotless suture anchor 14-3 may include an anchor body 48 and an eyelet 50. In this embodiment, the anchor body 48 and the eyelet 50 are relatively rigid plastic structures and thus the knotless suture anchor 14-3 is not considered to be a “soft” anchor assembly.

The anchor body 48 may be pre-loaded onto a driver 52. The anchor body 48 may be configured as a screw or an interference plug that is appropriately cannulated for receiving a shaft 54 of the driver 52. The eyelet 50 may be provided at a distal end 56 of the driver 52. The eyelet 50 may be releasably attached to the distal end 56 and may include an aperture 58 for receiving one or more sutures.

The anchor body 48 and the eyelet 50 of the knotless suture anchor 14-3 may be inserted into a socket 60 formed in the bone 12 (see FIG. 15). The socket 60 may be a preformed opening formed in the bone 12 that is configured for receiving the anchor body 48 and the eyelet 50. One of the suture tails 46 of the sutures 42 may be loaded through the eyelet 50, and then the eyelet 50 may be inserted into the socket 60. The suture tail 46 may then be tensioned prior to moving the anchor body 48 down toward the eyelet 50 within the socket 60. Once implanted within the socket 60, the anchor body 48 may trap the suture 42 between the bone 12 and the anchor body 48 in order to fixate the suture 42 in place. The knotless suture anchor 14-3 therefore provides the ability to implant the lateral row suture anchors 40 after connecting the sutures 42 to the surgical buttons 34 during the surgical method.

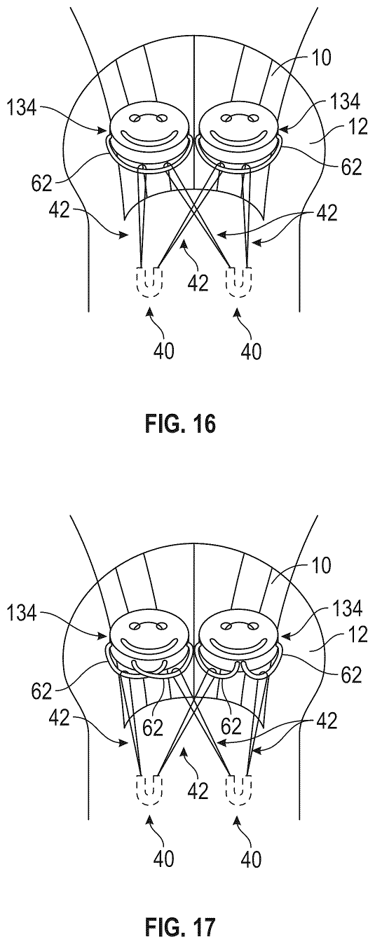

In the above embodiments, the sutures 42 of the lateral row of suture anchors 40 are connected to the surgical buttons 34 via openings formed through the surgical buttons 34 (e.g., via the third openings 44). However, other implantations of the surgical button are contemplated within the scope of this disclosure. For example, as shown in FIGS. 16-17, another exemplary surgical button 134 may include one or more suture loop constructs 62 that may be provided in place of the third opening 44 of the surgical button 34. In the embodiment of FIG. 16, the surgical buttons 134 each include a single suture loop construct 62. In the embodiment of FIG. 17, the surgical buttons 134 each include multiple suture loop constructs 62.

Each suture loop construct 62 may be attached or integrally formed with a body of its respective surgical button 134. The sutures 42 from the lateral row of suture anchors 40 may be looped over the suture loop constructs 62 either before or after implanting the lateral row of suture anchors 40 as part of the surgical method.

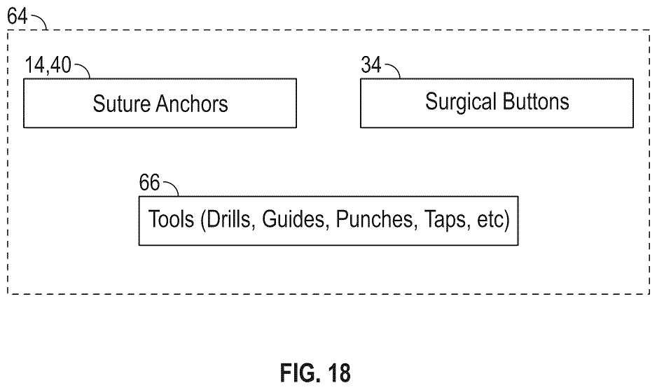

FIG. 18 schematically illustrates an exemplary tissue fixation system 64 that may be provided for performing the surgical method described above. The tissue fixation system 64 may be provided in the form of a surgical kit that includes all the necessary tools and components for performing surgical methods for reducing and reattaching torn tissue to bone. In an embodiment, the tissue fixation system 64 may include at least the following components:
1. At least (4) suture anchors 14, 40;
2. At least two surgical buttons 34; and
3. Various tools 66 (e.g., disposable drills, drill guides, punches, taps, etc.) for inserting the suture anchors 14, 40.

Other components or different combinations of components could be provided as part of the tissue fixation system 64 within the scope of this disclosure. For example, the tissue fixation system 64 could include various templates, scorers, curettes, and/or measuring devices that may be utilized to help prepare the tissue 10 and the bone 12 for performing the surgical methods discussed herein.

The tissue fixation systems and surgical methods described herein may be utilized to approximate, fixate, and compress tissue to bone. The proposed systems and methods provide a multi-point fixation configuration for fixating tissue to bone. The use of surgical buttons in combination with medial and lateral suture anchors provides a relatively stiff construct for maintaining footprint compression, thereby maximizing tissue-to-bone contact.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A surgical method, comprising:
inserting a first suture anchor into a bone;
passing a first suture of the first suture anchor through a tissue;
feeding the first suture through a surgical button;
securing the first suture relative to the surgical button,
inserting a second suture anchor into the bone at a position that is lateral to the first suture anchor;
feeding a second suture of the second suture anchor through the surgical button at a different location than the first suture; and
securing the second suture relative to the surgical button.

2. The surgical method as recited in claim 1, wherein the surgical button includes a first opening that is a first round hole, a second opening that is a second round hole, and a third opening that is a semi-circular or arch-shaped slot.

3. The surgical method as recited in claim 2, wherein the first round hole, the second round hole, and the semi-circular or arch-shaped slot are arranged in a smiley face pattern.

4. The surgical method as recited in claim 1, wherein securing the first suture relative to the surgical button includes:
splicing the first suture through itself to establish a mattress stitch that spans between a first opening and a second opening of the surgical button.

5. The surgical method as recited in claim 4, wherein securing the first suture relative to the surgical button further includes:
tensioning the first suture to pull the tissue laterally over top of the first suture anchor.

6. The surgical method as recited in claim 4, wherein securing the first suture relative to the surgical button further includes:
tensioning the first suture to firmly fixate the surgical button against the tissue.

7. The surgical method as recited in claim 1, wherein securing the second suture relative to the surgical button includes:
splicing the second suture through itself to establish a suture loop that is looped through the surgical button.

8. The surgical method as recited in claim 7, wherein securing the second suture relative to the surgical button further includes:
tensioning the second suture to reduce the second suture further into place and provide a desired area of footprint compression over top of the tissue.

9. The surgical method as recited in claim 1, wherein the first suture anchor is part of a medial row of suture anchors and the second suture anchor is part of a lateral row of suture anchors, and further wherein a crisscross suture pattern establishes a desired area of footprint compression over top of the tissue.

10. The surgical method as recited in claim 1, wherein the first suture anchor and the second suture anchor are comprised exclusively of soft, suture-based materials.

11. A surgical method, comprising:
inserting a first suture anchor into a bone;
passing a first suture of the first suture anchor through a tissue;
feeding the first suture through a surgical button;
tensioning the first suture to firmly fixate the surgical button against the tissue,
inserting a second suture anchor into the bone at a position that is lateral to the first suture anchor;
looping a second suture of the second suture anchor about a third suture of the surgical button; and
securing the second suture back to the second suture anchor.

12. The surgical method as recited in claim 11, wherein the third suture is a suture loop construct that is attached to the surgical button.

13. The surgical method as recited in claim 11, wherein the third suture is a suture loop construct that is integrally formed with the surgical button.

14. The surgical method as recited in claim 11, wherein tensioning the first suture includes:
splicing the first suture through itself to establish a mattress stitch that spans between a first opening and a second opening of the surgical button.

15. The surgical method as recited in claim 11, wherein tensioning the first suture pulls the tissue laterally over top of the first suture anchor.

16. The surgical method as recited in claim 11, wherein securing the second suture back to the second suture anchor includes:
splicing the second suture through itself to establish a suture loop that is looped around the third suture of the surgical button.

17. The surgical method as recited in claim 16, wherein securing the second suture back to the second suture anchor includes:
tensioning the second suture to reduce the second suture further into place and provide a desired area of footprint compression over top of the tissue.

18. The surgical method as recited in claim 11, comprising:
inserting a third suture anchor into the bone;
passing a fourth suture of the third suture anchor through the tissue;
feeding the fourth suture through a second surgical button; and
tensioning the fourth suture to firmly fixate the second surgical button against the tissue.

19. The surgical method as recited in claim 18, comprising:
inserting a fourth suture anchor into the bone at a position that is lateral to the third suture anchor;
looping a fifth suture of the fourth suture anchor about a sixth suture of the second surgical button; and
securing the fifth suture back to the fourth suture anchor.

20. A tissue repair system for repairing a tissue defect, comprising:
a suture anchor; and
a surgical button including a first opening configured as a first round hole, a second opening configured as a second round hole, and a third opening configured as a semi-circular or arch-shaped slot.
